(19) United States
(12) Patent Application Publication
(10) Pub. No.: US 2020/0005222 A1
(43) Pub. Date: Jan. 2, 2020

(54) DYNAMIC LOAD OPTIMIZATION

(71) Applicant: SAP SE, Waldorf (DE)

(72) Inventor: Ran Bittmann, Tel Aviv (IL)

(21) Appl. No.: 16/020,794

(22) Filed: Jun. 27, 2018

(51) Int. Cl. G06Q 10/06; G16H 40/20

(52) U.S. Cl. CPC G06Q 10/063116; G16H 40/20

(57) ABSTRACT

A system for wireless communication is provided. In some implementations, the system performs operations including receiving activity data from a plurality of sensors associated with a plurality of workers, the activity data comprising location information of the plurality of sensors within an area and a quantity of steps walked by the plurality of workers. The operations further include determining, based on activity data received from a first sensor associated with a first worker, whether a first activity level of the first worker has satisfied a first activity threshold. The operations further include updating, in response to the determining, a schedule of a second worker. The operations further include sending a message indicating the updated schedule to the second sensor.

### TECHNICAL FIELD

The subject matter described herein relates to optimizing load balancing in a wireless network.

### BACKGROUND

Physical venues may include a workforce that includes individuals and groups in different locations. Certain events occurring at the venue may affect a workload distribution among different groups or departments at the venue.

### SUMMARY

Systems, methods, and articles of manufacture, including computer program products, are provided for data management. In one aspect, there is provided a system. The system may include at least one data processor and at least one memory. The at least one memory may store instructions that result in operations when executed by the at least one data processor. The operations may include receiving activity data from a plurality of sensors associated with a plurality of workers, the activity data comprising location information of the plurality of sensors within an area and a quantity of steps walked by the plurality of workers. The operations further include determining, based on activity data received from a first sensor associated with a first worker, whether a first activity level of the first worker has satisfied a first activity threshold. The operations further include updating, in response to the determining, a schedule of a second worker. The updating includes determining, based on activity data received from a second sensor associated with the second worker, a second activity level of the second worker. The updating further includes comparing the second activity level to a second activity threshold. The updating further includes assigning, based on the comparing, the second worker to a location associated with the first sensor. The operations further include sending a message indicating the updated schedule to the second sensor.

In another aspect, there is provided a method. The method includes receiving, at a processor, activity data from a plurality of sensors associated with a plurality of workers, the activity data comprising location information of the plurality of sensors within an area and a quantity of steps walked by the plurality of workers. The method further includes determining, by the processor and based on activity data received from a first sensor associated with a first worker, whether a first activity level of the first worker has satisfied a first activity threshold. The method further includes updating, by the processor and in response to the determining, a schedule of a second worker. The updating includes determining, based on activity data received from a second sensor associated with the second worker, a second activity level of the second worker. The updating further includes comparing the second activity level to a second activity threshold. The updating further includes assigning, based on the comparing, the second worker to a location associated with the first sensor. The method further includes sending, by the first processor, a message indicating the updated schedule to the second user equipment.

In another aspect, there is provided a non-transitory computer program product storing instructions which, when executed by at least one data processor, causes operations which include receiving activity data from a plurality of sensors associated with a plurality of workers, the activity data comprising location information of the plurality of sensors within an area and a quantity of steps walked by the plurality of workers. The operations further include determining, based on activity data received from a first sensor associated with a first worker, whether a first activity level of the first worker has satisfied a first activity threshold. The operations further include updating, in response to the determining, a schedule of a second worker. The updating includes determining, based on activity data received from a second sensor associated with the second worker, a second activity level of the second worker. The updating further includes comparing the second activity level to a second activity threshold. The updating further includes assigning, based on the comparing, the second worker to a location associated with the first sensor. The operations further include sending a message indicating the updated schedule to the second sensor.

In some variations, one or more features disclosed herein including the following features may optionally be included in any feasible combination. The area may include a medical facility. The first worker may be associated with a first group having a first location in the medical facility. The second worker may be associated with a second group having a second location of the medical facility. The first location may be different than the second location. The determining may include comparing a quantity of steps over a time period to a step threshold. The quantity of steps may be associated with the first sensor. The assigning may be based further on a location of the second sensor in relation to the first sensor. The operations and/or method may further include transmitting a notification to the first sensor, the notification comprising a rest time period. The first sensor may be associated with a first group and the second sensor may be associated with a second group, the assigning may be based further on a quantity of sensors in the second group. The assigning may be based further on a quantity of sensors in the first group. The assigning may be based further on a profile associated with the second sensor and/or the second group. The operations and/or method may further include monitoring, in response to the updating, the second sensor; and updating, based on the monitoring, the first activity threshold and/or the second activity threshold. The updating of the first activity threshold and/or the second activity threshold may be further based on a machine learning model. The machine learning model may be trained to determine optimized schedules for the plurality of workers based on training activity data such as activity data received from the plurality of sensors The machine learning model may include a neural network. In some aspects, the receiving, the determining, the updating the schedule of the second sensor, and/or the sending, may be performed by the neural network.

Implementations of the current subject matter may include methods consistent with the descriptions provided herein as well as articles that include a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which may include a non-transitory computer-readable or machine-readable storage medium, may include, encode, store, or the like one or more programs that cause one or more processors to perform one or more of the operations described herein. Computer implemented methods consistent with one or more implementations of the current subject matter may be implemented by one or more data processors residing in a single computing system or multiple computing systems. Such multiple computing systems may be connected and may exchange data and/or commands or other instructions or the like via one or more connections, including, for example, to a connection over a network (e.g. the Internet, a wireless wide area network, a local area network, a personal area network (PAN), a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to a wireless network, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts an example of a system for controlling workload, in accordance with some example embodiments;

FIG. 2 depicts an example of a process for load optimization, in accordance with some example embodiments;

FIG. 3 depicts a block diagram illustrating a computing apparatus, in accordance with some example implementations; and

FIG. 4 depicts another example of a process for load optimization, in accordance with some example implementations.

When practical, similar reference numbers denote similar structures, features, or elements.

### DETAILED DESCRIPTION

Many workplaces employ staff that frequently attend to emerging issues. An example for such a workplace is a hospital where there are different staff members, such as maintenance workers, nurses, and doctors. These workers may be distributed into departments with a workforce of similar skill, who attend issues both in a routine level, like scheduled patient visits, and event based issues, like emergencies or occasional patient assistance.

During the course of the day, which can be very hectic, it may be difficult to keep track of the workload of the staff and its utilization may not be ideal. While some employees might be underutilized, others may be overloaded. In addition, there is no recording of the actual workload distribution. Employers and companies may want to adjust workload for its staff based on the changing needs of different departments, events, times of day, and/or the like.

In some example embodiments, the locations, and/or other physiological data, of one or more groups of user equipment carried by the individual staff workers may be monitored as the staff perform their work functions. Moreover, the workloads associated with different individual and/or groups of the staff may be controlled in order to optimize a certain set of criteria, such as minimization of patient/client wait times, encouraging rest, maximizing the number of staff workers or a certain group of staff to a particular location, and/or the like. The optimized workload may thus provide a scheduled time for a group of workers to move from one location to another, to take a break, to assist other staff, and/or the like. The workload may be optimized on a group level and across some if not all groups/departments, in accordance with some example embodiments. In addition, the workload optimization may be performed dynamically in the sense that as conditions change at the venue, such as spikes in workload activity due to an event, the workload optimization may be updated.

Although some of the examples described herein refer to workload optimization in a hospital, the workload optimization disclosed herein may be used to control the workload in any other type of venue or area including a factory, a construction site, an airport, and/or the like. Moreover, although some of the examples refer to movement trackers, such as found in smart phones or fitness trackers, other tracking technology may be implemented. These tracking devices may transmit in real-time the activity of their wearer using protocols such as ANT+, Bluetooth, Wi-Fi, and/or the like.

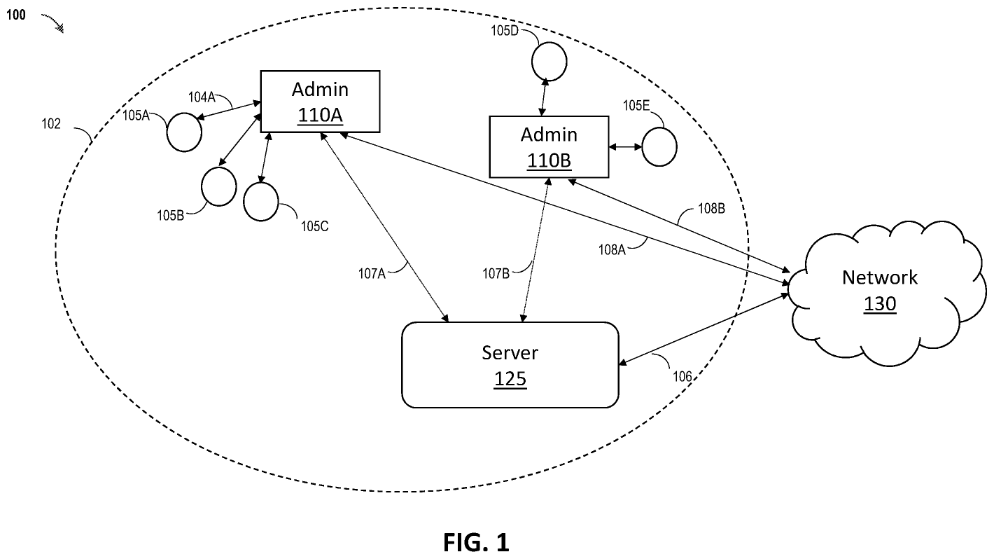

FIG. 1 depicts an example system 100, in accordance with some example embodiments. The system 100 may include a plurality of user equipment 105A-E, 110A-B, and a server 125 within a wireless coverage area 102. This coverage area may represent some, if not all, of the geographic area associated with a venue, such as an office building, a hospital, factory, and/or the like.

In the example of FIG. 1, user equipment 105A-E may correspond to devices being tracked within a certain coverage area or zone 102 in the venue. As such, the user equipment 105A-E may also be referred to herein as “workers” or “staff” Although the user equipment 105A-E may be registered with a specific user/worker, the user equipment 105A-E may be associated with the worker's function to maintain privacy. For example, in a hospital setting, different functions may include nurses, doctors, researchers, administrative workers, facility workers, and/or the like. Each of user equipment 110A-B may represent a local administrator which may monitor a particular coverage area and/or a set or workers within the coverage area, such as a particular department, specialty group, floor, and/or the like. The server 125 may represent a server configured to receive sensor information, such as movement, distance, biometric data, speed, physiological data, and/or the like from workers 105 and/or administrators 110.

In the example of FIG. 1, administrator 110A and workers 105A-C may represent a first group, while administrator 110B and workers 105D-E may represent a second group. For example, the first group may represent nurses, while the second group may represent a group of doctors. One or more groups may be aggregated to form a “network,” within a predefined or dynamic geographical region, such as coverage area 102. The network may represent for example a defined collection of groups, such as a collection of groups defined by a certain specialty (e.g., radiology, emergency room, neurology, etc.). Although this example refers to a group having a single administrator 110, and a plurality of workers 105, the group may use other quantities of administrators 110 and workers 105 as well. For example, a group may have a plurality of administrators 110 as well. Moreover, a group may be formed with a single administrator 110 and/or a single worker 105. Although the previous examples refer to two groups, other quantities of groups may be implemented as well.

In some example embodiments, a group of workers such as workers 105A-C may be associated with an administrator 110A. The group of workers 105A-C may provide location information as well as other information to their associated administrator 110A (and/or directly to the server 125 as well). The administrator 110A may then provide the group's information to the server 125, which performs the workload optimization. As such, server 125 may send to administrator 110A and/or workers 105A-C an optimized workload distribution in the form of a revised schedule for one or more of the workers 105A-C. The revised schedule may include a re-assignment to another group or location, a rest period, a revised shift schedule, and/or the like.

Moreover, as the state of the venue changes due to for example a temporary spike in activity in one area, such as a spike in personnel needed in the emergency room (ER) due to an accident, the server 125 may send an update message/notification including a revised workload. The update message may include, a message for one or more workers from a group or groups to assist the current workers in the ER. For example, the server 125 may be configured to communicate information related to the workers 105 and/or administrators 110 a dashboard of a user interface that may display in real-time, current workload of the different workers 105, administrators 110 and the functions associated with each. The server 125 may notify workers 105 and/or administrators 110 via the user interface and/or via the user equipment 105 and/or 110. The server 125 may monitor the activity in each department or group of the workforce and provide alerts and/or recommendations in real-time regarding an optimized workload distribution. For example, when there is a crisis in a first department, the server 125 may notify a second department where there is more moderate activity, to assist the first department.

Although the previous example describes workload optimization being performed by the server 125, the administrator 110 may also perform workload optimization. For example, the server 125 may provide criteria and other information to the administrator 110 to enable the administrator 110 to optimize the workload for the group. In this example, the administrator 110 may report the optimized workload to the server 125.

In some example embodiments, the user equipment 105 may be configured to determine its location, provide the obtained location information to other devices such as an administrator 110 (and/or the server 125), provide user equipment 105 status information to the other devices, display a workload schedule for a worker (for example, a shift schedule for a given group or department), provide other biometric/physiological data associated with a worker (for example, a quantity of steps walked/run, a heart rate, a blood pressure measurement, a breathing rate, a sweat rate, a body temperature, etc.) and/or the like. The location information may comprise a latitude and longitude of the user equipment 105 and/or a timestamp, although other forms of location information may be used as well (for example, floor number, department number, and/or the like). The user equipment 105 status information may include whether the user equipment 105 is on or off, amount of battery remaining at the user equipment 105, a current capability of the user equipment 105, and/or any other information that may be used by the system and/or server 125.

In some example embodiments, the worker 105 may provide information to an administrator 110 directly via a direct wireless link, or may provide information indirectly via another device or another network (for example, via network 130, such as the Internet, a cellular network, a Wi-Fi network, ANT+, Bluetooth, and/or the like). For example, worker 105A may transmit location information via link 104A to administrator 110A. The point-to-point link 104A may comprise a Wi-Fi direct, Bluetooth, Bluetooth low energy, ANT+, and/or any other type of radio technology. Alternatively or additionally, worker 105A may send a message to administrator 110A indirectly via a cellular network, Wi-Fi network, the Internet, a PAN, and/or the like. The workers 105 may provide location information to the administrator 110 or server 125 from time to time, continuously, and/or upon request.

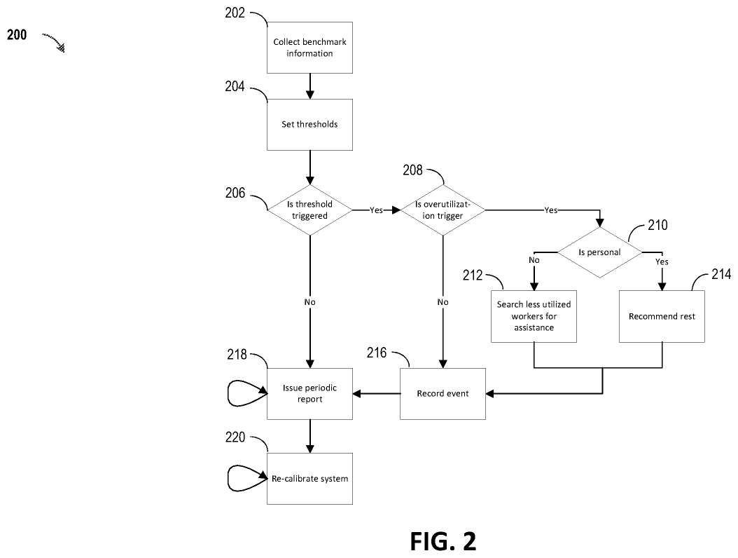

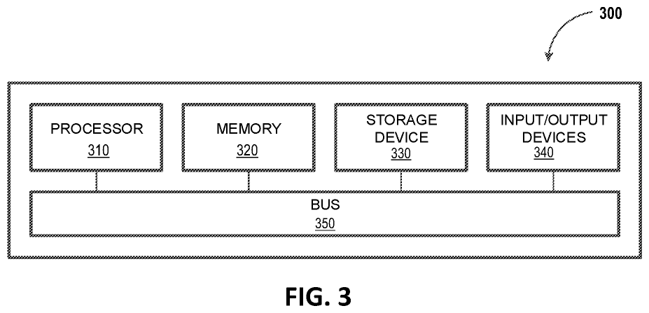

FIG. 2 depicts an example of a process 200 for load optimization, in accordance with some example embodiments. Referring to FIGS. 1 and 3, the process 200 may be performed by a computing apparatus such as the administrator 110, the server 125, and/or the computing apparatus 300.

At operational block 202, the server 125, for example, may collect benchmark information for calibrating a system, such as the system 100. The benchmark information may include information associated with individual workers 105 and/or groups of workers 105. The information may include a quantity of steps, an average number of steps, a quantity of stairs/floors climbed, a gait analysis/measurement, a rate of steps per time period, pattern recognition for locations traveled to or paths taken, a heart rate, a distribution of workers over an average shift, an average number of workers in a department or location for a time period, and/or the like. The time period for collecting the benchmark information may be defined by the server 125, an administrator 110, and/or the system 100. In some aspects, the time period may be modified.

At operational block 204, the server 125, for example, may set threshold values for triggering a modification to one or more workers 105 workload schedule. Example thresholds may include a threshold of 30% above average activity to identify an over-utilized worker 105 or group, and a threshold of 20% below average activity to identify an underutilized worker 105 or group, although other thresholds may be established as well. Another example for threshold may be a number of standard deviations away from the mean calculated using the collected benchmark in formation. For example, a threshold for a given distribution of a parameter, such as quantity of steps walked, may be 3 standard deviations above the mean for signifying over-utilization or 2 standard deviations below the mean for signifying under-utilization. The thresholds may be associated with a given group or groups in a department or departments, and may also be associated with an individual worker 105. In some aspects, individual worker 105 information may not be shared in the server 125 for privacy or regulatory compliance reasons.

At operational block 206, the server 125, for example, may determine whether a threshold has been satisfied or triggered. The server 125 may monitor the activity of the workers 105 and/or administrators 110 and determine whether one or more thresholds have been satisfied based on the monitoring. In some aspects, the server 125 may compare metrics received from the workers 105 and/or administrators 110 to the one or more thresholds. When a threshold is satisfied, an event may be triggered. For example, the server 125 may receive, from the workers 105A-C and/or the administrator 110A, a quantity of steps walked by the workers 105A-C in the last hour. The workers 105A-C may represent nurses in the ER department of a hospital. The server 125 may compare the received quantity of steps to a threshold for the workers 105A-C, such as 30% above, or 20% below, an average quantity of steps per hour. If the threshold has been satisfied, the server 125 may trigger an event. The event may include a workload analysis of the workers 105 in communication with the server 125, such as workers 105A-E.

At operational block 208, the server 125, for example, may determine whether an over-utilization threshold has been satisfied or triggered. If yes, then the process 200 may proceed to operational block 210. If the threshold was not satisfied, the process 200 may proceed to operational block 216. For example, the server 125 may determine that the quantity of steps walked by the workers 105A-C in the last hour exceed the average quantity of steps per hour for the group by 50%. As such, the server 125 may determine that the threshold triggered in operational block 206 was an over-utilization threshold.

At operational block 210, the server 125, for example, may determine whether the over-utilization threshold relates to an individual or to a group. If the over-utilization is not related to an individual worker 105, then the threshold may relate to a group of workers 105 and/or administrators 110 and the process 200 may proceed to operational block 212. In the example above, the over-utilization threshold triggered relates to the workers 105A-C, a group of nurses in the ER department. If the over-utilization is related to an individual worker 105, such as a single nurse 105A in the ER department, then the process 200 may proceed to operational block 214.

At operational block 212, the server 125, for example, may search less utilized workers 105 to assist the over-utilized group. In some aspects, the server 125 may determine which workers 105 to notify based on certain criteria. For example, the server 125 may base its determination on the location of the workers 105, whether a worker 105 satisfies an under-utilization threshold, an average utilization of the worker 105 for a week or other time period, a total number of workers 105 in a group, qualifications of the worker 105, a profile of the worker 105, and/or the like. The server may prioritize which workers 105 and/or groups to notify based on the event or other criteria. For example, if an emergency room activity spikes up, the server 125 may prioritize the closest workers 105 to assist the ER. In other aspects, if a cardiologist department is over-utilized, the server 125 may prioritize certain workers 105 with a cardiology background or experience. Referring to FIG. 1, the server 125 may receive, from the workers 105D-E and/or the administrator 110B, a quantity of steps walked by the workers 105D-E in the last hour. The workers 105D-E may represent nurses in an operating room (OR) department of the hospital. The server 125 may determine, based on the quantity of steps or a location of the workers 105D-E, to notify the workers 105D-E to assist the nurses 105A-C in the ER department. In some aspects, the server 125 may notify the workers 105D-E based on the workers 105D-E satisfying an under-utilization threshold or the workers 105A-C exceeding the overutilization threshold by a certain amount, such as 50% above the over-utilization threshold.

At operational block 214, the server 125, for example, may recommend rest for individual work 105 that was determined to be over-utilized or overworked. Additionally, the server 125 may also search for a second worker 105 to replace the over-utilized worker 105. Like the determination in operational block 212, the server 125 may base its search on various criteria depending on the over-utilized worker 105, the event, the workers 105 available, or other factors. In some aspects, the server 125 may notify the over-utilized worker 105 directly, through an administrator 110, through a user interface, and/or the like.

At operational block 216, the server 125, for example, may record information relating to the event or events that triggered the over-utilization. The information recorded may be stored in a cloud or external server accessible by the server 125 or in a database of the server 125. For example, the information may include a time of day of the event, a quantity of workers 105 involved, a function of the workers 105, a location of the event, a quantity of steps walked during the event, a function of the workers 105 notified to assist, a location of the workers 105 notified to assist, a reduction in worker utilization after notifying other workers 105 to assist, and/or the like.

At operational block 218, the server 125, for example, may issue periodic reports. The periodic reports may include capacity planning based on the information gathered during historic events. In some aspects, the capacity planning can provide recommendations to administrators 110 or other employees of a company regarding workload distribution and staffing. For example, the recommendations may identify chronic alerts in the system 100 as an understaffing/overstaffing issue and/or qualifications issue. The periodic reports may also include updated threshold values and/or criteria and may archive recorded information for future use.

At operational block 210, the server 125, for example, may re-calibrate the system 100 based on information collected during the event or over a certain operational time period. The server 125 may collect information (e.g., schedules, distance, speed average, standard deviations, etc.) based on actual events and the collected and/or calculated metrics may be optimized using machine learning models. The server 125 may then adjust thresholds, re-assignment methods and/or criteria, rest periods, staff schedules, and/or the like based on the collected information and/or the machine learning model. For example, the server 125 may permanently assign the worker 105D to the ER department with workers 105A-C based on a consistent pattern of over-utilization of workers 105A-C and/or a consistent pattern of underutilization for the worker 105D. Additionally, the server 125 determine that the average number of steps for the workers 105A-C has increased since the benchmark collection period and may accordingly increase the over-utilization threshold value and/or the underutilization threshold value associated with the group of workers 105A-C.

In some aspects, the machine learning model may include a Support Vector Machine (SVM), neural networks, Naïve Bayes, and/or the like to calculate optimized features that may determine optimized schedules for the plurality of workers, predict a disruptive event, and/or improve reactions to the disruptive event. For example, the machine learning model may analyze the benchmark information and other system information to determine that an over-utilization threshold in a first department (e.g., the ER department) may lead to an over-utilization of a second department (e.g., the OR department) 30 minutes, or some other time period, after the over-utilization threshold is triggered in the first department. The server 125, based on the machine model, may then allocate extra workers 105 to the second department when the over-utilization threshold is triggered at the first department to improve workload balance as a result of an event.

The process 200 may improve workflow and efficiency of wireless systems. The system 100 may efficiently monitor activity and analyze large amounts of data to optimize workload across the system. Additionally, the system 100 may utilize feedback and machine learning to improve optimization as new events occur that could potentially disrupt optimal workflow.

FIG. 3 depicts a block diagram illustrating a computing apparatus 400 consistent with implementations of the current subject matter. Referring to FIGS. 1, 2 and 4, the computing apparatus 300 may be used to implement at least a portion of the server 125 and/or any components therein.

As shown in FIG. 3, the computing apparatus 300 may include a processor 310, a memory 320, a storage device 330, and input/output devices 340. The processor 310, the memory 320, the storage device 330, and the input/output devices 340 may be interconnected via a system bus 350. The processor 310 is capable of processing instructions for execution within the computing apparatus 300. Such executed instructions may be implement by one or more components of, for example, the server 125. In some example implementations, the processor 310 may be a single-threaded processor. Alternately, the processor 310 may be a multi-threaded processor. The processor 310 is capable of processing instructions stored in the memory 320 and/or on the storage device 330 to display graphical information for a user interface provided via the input/output device 340.

The memory 320 is a computer readable medium such as volatile or non-volatile that stores information within the computing apparatus 300. The memory 320 may store data structures representing configuration object databases, for example. In some aspects, the memory 320 may include a cache memory and/or a main memory. The storage device 330 is capable of providing persistent storage for the computing apparatus 300. The storage device 330 may be a floppy disk device, a hard disk device, an optical disk device, or a tape device, or other suitable persistent storage means. The input/output device 340 provides input/output operations for the computing apparatus 300. In some example implementations, the input/output device 340 includes a keyboard and/or pointing device. In various implementations, the input/output device 340 includes a display unit for displaying graphical user interfaces.

According to some example implementations, the input/output device 340 may provide input/output operations for a network device. For example, the input/output device 340 may include Ethernet ports or other networking ports to communicate with one or more wired and/or wireless networks (e.g., a local area network (LAN), a wide area network (WAN), the Internet). The input/output device 340 may include one or more antennas for communication over the network 130 with the user equipment 105 and 110, and the server 125.

In some example implementations, the computing apparatus 300 may be used to execute various interactive computer software applications that may be used for organization, analysis and/or storage of data in various formats. Alternatively, the computing apparatus 300 may be used to execute any type of software applications. These applications may be used to perform various functionalities, e.g., planning functionalities (e.g., generating, managing, editing of spreadsheet documents, word processing documents, and/or any other objects, etc.), computing functionalities, communications functionalities, etc. The applications may include various add-in functionalities or may be standalone computing products and/or functionalities. Upon activation within the applications, the functionalities may be used to generate the user interface provided via the input/output device 340. The user interface may be generated and presented to a user by the computing apparatus 300 (e.g., on a computer screen monitor, etc.).

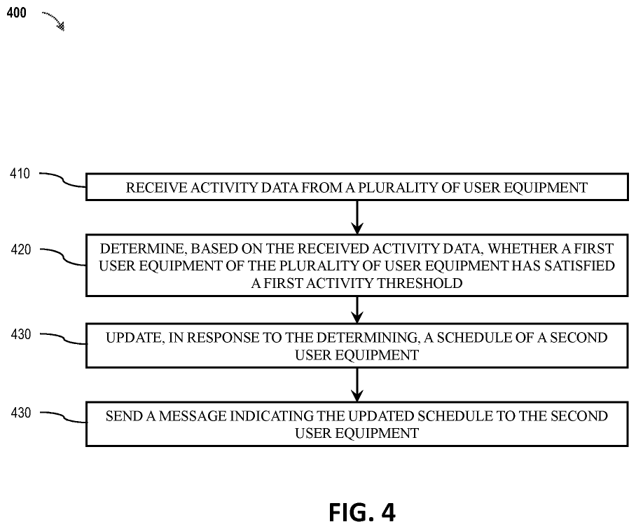

FIG. 4 depicts another example of a process 400 for load optimization, in accordance with some example implementations. Referring to FIGS. 1-4, the process 400 may be performed by a computing apparatus such as, the administrator 110, the server 125, and/or the computing apparatus 300.

At operational block 410, the computing apparatus 300, for example, may receive activity data from a plurality of sensors. The activity data may include location information of the plurality of sensors within an area and a quantity of steps walked by the plurality of workers. At operational block 420, the computing apparatus 300, for example, may determine, based on activity data received from a first sensor associated with a first worker, whether a first activity level of the first worker has satisfied a first activity threshold. In the example above, the computing apparatus 300 may receive, from the workers 105A-C and/or the administrator 110A, a quantity of steps walked by the ER nurse workers 105A-C in the last hour. The computing apparatus 300 may compare the received quantity of steps to the first activity threshold for the workers 105A-C, such as 30% above, or 20% below, an average quantity of steps per hour.

At operational block 430, the computing apparatus 300, for example, may update, in response to the determining, a schedule of a second worker. The updating may include determining, based on activity data received from a second sensor associated with the second worker, a second activity level of the second worker. The updating may further include comparing the second activity level to a second activity threshold, and assigning, based on the comparing, the second worker to a location associated with the first sensor. Referring to FIGS. 1 and 3, the computing apparatus 300 may determine, in response to the workers 105A-C triggering the over-utilization threshold, to update a schedule the OR nurse workers 105D-E. Updating the schedule may include assigning the workers to 105D-E to the ER room location to assist the nurses 105A-C in the ER department. The updated schedule may include a time and/or a location for the workers 105D-E to assist the workers 105A-C. In some implementations, the computing apparatus 300 may base the updating on a quantity of steps walked by the OR nurse workers 105D-E in the last hour, a location of the workers 105D-E, a qualification of the workers 105D-E, and/or the like. At operational block 440, the computing apparatus 300, for example, may send a message indicating the updated schedule to the second sensor. For example, the computing apparatus 300 may transmit the message directly to the workers 105D-E to notify them of the updated schedule. In another example, the computing apparatus may send the message to the administrator 110B for the administrator 110B to notify the workers 105D-E of the updated schedule.

One or more aspects or features of the subject matter described herein may be realized in digital electronic circuitry, integrated circuitry, specially designed ASICs, field programmable gate arrays (FPGAs) computer hardware, firmware, software, and/or combinations thereof. These various aspects or features may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which may also be referred to as programs, software, software applications, applications, components, or code, include machine instructions for a programmable processor, and may be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium may store such machine instructions non-transitorily, such as for example as would a non-transient solid-state memory or a magnetic hard drive or any equivalent storage medium. The machine-readable medium may alternatively or additionally store such machine instructions in a transient manner, such as for example, as would a processor cache or other random access memory associated with one or more physical processor cores.

To provide for interaction with a user, one or more aspects or features of the subject matter described herein may be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices may be used to provide for interaction with a user as well. For example, feedback provided to the user may be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein may be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations may be provided in addition to those set forth herein. For example, the implementations described above may be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method comprising:
receiving, at a processor, activity data from a plurality of sensors associated with a plurality of workers, the activity data comprising location information of the plurality of sensors within an area and a quantity of steps walked by the plurality of workers;
determining, by the processor and based on activity data received from a first sensor associated with a first worker, whether a first activity level of the first worker has satisfied a first activity threshold;
updating, by the processor and in response to the determining, a schedule of a second worker, the updating comprising:
determining, based on activity data received from a second sensor associated with the second worker, a second activity level of the second worker,
comparing the second activity level to a second activity threshold, and
assigning, based on the comparing, the second worker to a location associated with the first sensor; and
sending, by the processor, a message indicating the updated schedule to the second sensor.

2. The method of claim 1, wherein the area comprises a medical facility, wherein the first worker is associated with a first group having a first location in the medical facility, wherein the second worker is associated with a second group having a second location of the medical facility, and wherein the first location is different than the second location.

3. The method of claim 1, wherein the assigning is based further on a location of the second sensor in relation to the first sensor.

4. The method of claim 1, further comprising:
transmitting a notification to the first sensor, the notification comprising a rest time period.

5. The method of claim 1, wherein the first sensor is associated with a first group and the second sensor is associated with a second group, and wherein the assigning is based further on a quantity of sensors in the second group.

6. The method of claim 1, wherein the determining comprises comparing, to a step threshold, a quantity of steps associated with the first worker over a time period.

7. The method of claim 5, wherein the assigning is based further on a profile associated with the second worker and/or the second group, and wherein the profile comprises a function of the second worker and/or the second group.

8. The method of claim 1, further comprising:
monitoring, by the processor and in response to the updating, the second sensor; and
updating, by the processor and based on the monitoring, the first activity threshold and/or the second activity threshold.

9. The method of claim 8, wherein the updating of the first activity threshold and/or the second activity threshold is performed by a machine learning model, and wherein the machine learning model is trained to determine, based on the activity data, optimized schedules for the plurality of workers.

10. The method of claim 9, wherein the machine learning model comprises a neural network and/or a support vector machine.

11. A system, comprising:
at least one data processor; and
at least one memory storing instructions which, when executed by the at least one data processor, result in operations comprising:
receiving activity data from a plurality of sensors associated with a plurality of workers, the activity data comprising location information of the plurality of sensors within an area and a quantity of steps walked by the plurality of workers;
determining, based on activity data received from a first sensor associated with a first worker, whether a first activity level of the first worker has satisfied a first activity threshold;
updating, in response to the determining, a schedule of a second worker, the updating comprising:
determining, based on activity data received from a second sensor associated with the second worker, a second activity level of the second worker,
comparing the second activity level to a second activity threshold, and
assigning, based on the comparing, the second worker to a location associated with the first sensor; and
sending a message indicating the updated schedule to the second sensor.

12. The system of claim 11, wherein the area comprises a medical facility, wherein the first worker is associated with a first group having a first location in the medical facility, wherein the second worker is associated with a second group having a second location of the medical facility, and wherein the first location is different than the second location.

13. The system of claim 11, wherein the assigning is based further on a location of the second sensor in relation to the first sensor.

14. The system of claim 11, wherein the operations further comprise:
transmitting a notification to the first sensor, the notification comprising a rest time period.

15. The system of claim 11, wherein the first sensor is associated with a first group and the second sensor is associated with a second group, wherein the assigning is based further on a quantity of sensors in the second group.

16. The system of claim 15, wherein the determining comprises comparing, to a step threshold, a quantity of steps associated with the first worker over a time period.

17. The system of claim 15, wherein the assigning is based further on a profile associated with the second worker and/or the second group, and wherein the profile comprises a function of the second worker and/or the second group.

18. The system of claim 11, wherein the operations further comprise:
monitoring, in response to the updating, the second sensor; and
updating, based on the monitoring, the first activity threshold and/or the second activity threshold.

19. The system of claim 18, wherein the updating of the first activity threshold and/or the second activity threshold is performed by a machine learning model, and wherein the machine learning model is trained to determine, based on the activity data, optimized schedules for the plurality of workers.

20. A non-transitory computer program product storing instructions which, when executed by at least one data processor, causes operations comprising:
receiving activity data from a plurality of sensors associated with a plurality of workers, the activity data comprising location information of the plurality of sensors within an area and a quantity of steps walked by the plurality of workers;
determining, based on activity data received from a first sensor associated with a first worker, whether a first activity level of the first worker has satisfied a first activity threshold;
updating, in response to the determining, a schedule of a second worker, the updating comprising:
determining, based on activity data received from a second sensor associated with the second worker, a second activity level of the second worker,
comparing the second activity level to a second activity threshold, and
assigning, based on the comparing, the second worker to a location associated with the first sensor; and
sending a message indicating the updated schedule to the second sensor.
